(19) United States
(12) Patent Application Publication
(10) Pub. No.: US 2018/0353893 A1
(43) Pub. Date: Dec. 13, 2018

(54) REMOVAL OF HETEROATOM-CONTAINING COMPOUNDS FROM FLUIDS

(71) Applicant: William Marsh Rice University, Houston, TX (US)

(72) Inventors: Mayank Gupta, Houston, TX (US); Priscilla Dias da Silva, Brasilia, DF (BR); Scott L. Wellington, Bellaire, TX (US); Michael S. Wong, Houston, TX (US); Kyriacos Zygourakis, Houston, TX (US)

(73) Assignee: William Marsh Rice University, Houston, TX (US)

(21) Appl. No.: 16/061,126

(22) PCT Filed: Dec. 9, 2016
(86) PCT No.: PCT/US16/65888

§ 371 Date: Jun. 11, 2018

(51) Int. Cl. B01D 53/02; B01D 15/18; B01D 53/86; C07C 7/13; C10G 25/05; B01J 20/18; B01J 20/28

(52) U.S. Cl. CPC B01D 53/02; C10G 2300/202; B01D 53/8603; B01D 53/8621; C07C 7/13; C10G 25/05; B01J 20/186; B01J 20/28059; B01J 20/28061; B01J 20/28064; B01D 2253/108; B01D 2253/1122; B01D 2253/25; B01D 2255/104; B01D 2255/20761; B01D 2255/20746; B01D 2255/2027; B01D 2255/2061; B01D 2255/20753; B01D 2255/9207; B01D 2255/50; B01D 2256/24; B01D 2257/306; B01D 2257/304; B01D 2257/40; B01D 15/1871

(57) ABSTRACT

In some embodiments, the present disclosure pertains to methods of removing heteroatoms from a fluid by associating the fluid with one or more adsorbents, where the association results in the removal of the heteroatoms from the fluid. The association may occur by associating the fluid with a single adsorbent or a plurality of adsorbents in a sequential manner that maximizes heteroatom removal efficacy. The methods may be utilized to remove heteroatom-containing compounds from various fluids, such as fuels, hydrocarbons, alcohols, water, organic solvents, and combinations thereof. The one or more adsorbents may include, without limitation, activated carbon, zeolites, ion exchanged zeolites, ion impregnated zeolites, alumina, alumina nanowires, carbon-based supports, and combinations thereof. The methods of the present disclosure can be utilized to reduce heteroatoms in the fluid by more than about 50%, by more than about 80%, or by more than about 99%.

### CROSS-REFERENCE TO RELATED APPLICATIONS

This application claims priority to U.S. Provisional Patent Application No. 62/265,241, filed on Dec. 9, 2015. The entirety of the aforementioned application is incorporated herein by reference.

### STATEMENT REGARDING FEDERALLY SPONSORED RESEARCH

Not applicable.

### BACKGROUND

Current methods of removing heteroatom compounds from fluids have numerous limitations. The present disclosure addresses such limitations.

### SUMMARY

In some embodiments, the present disclosure pertains to methods of removing heteroatoms from a fluid by associating the fluid with one or more adsorbents. The association results in the removal of the heteroatoms from the fluid.

The association of fluids with one or more adsorbents can occur in various manners. For instance, in some embodiments, the association occurs by contacting the fluid with one or more adsorbents. In some embodiments, the association occurs by associating the fluid with a single adsorbent. In some embodiments, the association occurs in a single step or in multiple steps. In some embodiments, the association occurs by associating the fluid with a plurality of adsorbents in a sequential manner that maximizes heteroatom removal efficacy.

Various heteroatoms may be removed from various fluids. For instance, in some embodiments, the heteroatoms to be removed include heteroatom-containing compounds such as sulfur-containing compounds, nitrogen-containing compounds, oxygen-containing compounds, and combinations thereof. In some embodiments, the fluids that contain the heteroatoms to be removed include, without limitation, fuels, hydrocarbons, alcohols, water, organic solvents, and combinations thereof.

Various adsorbents may be utilized to remove heteroatoms from fluids. For instance, in some embodiments, one or more adsorbents of the same kind may be utilized. In some embodiments, different kinds of adsorbents may be utilized. In some embodiments, the one or more adsorbents include, without limitation, activated carbon, zeolites, ion exchanged zeolites, ion impregnated zeolites, alumina, alumina nanowires, carbon-based supports, and combinations thereof. In some embodiments, the one or more adsorbents include additional components, such as active metals, transition metals, oxides thereof, sulfides thereof, and combinations thereof.

In some embodiments, one or more adsorbent components are affixed to a solid support, such as alumina, alumina nanowires, activated carbon, zeolites, and combinations thereof. In some embodiments, the one or more adsorbents include ion exchanged zeolites or ion impregnated zeolites.

The methods of the present disclosure can be utilized to remove various amounts of heteroatoms from samples. For instance, in some embodiments, the methods of the present disclosure can be utilized to reduce the heteroatom content of a fluid to below 30 ppmw. In some embodiments, the methods of the present disclosure can be utilized to reduce the heteroatom content of a fluid to below 10 ppmw. In some embodiments, the methods of the present disclosure can be utilized to reduce heteroatoms in the fluid by more than about 50%, by more than about 80%, or by more than about 99%.

### FIGURES

FIG. 1 provides a scheme of a method of removing heteroatoms from a fluid.

FIG. 2 provides a scheme of a desulfurization series for 3% Ag impregnated Na—Y Zeolite. Each arrow shows the sulfur content in the fluid (i.e., JP-8) in parts per million (ppm).

FIG. 3 provides a scheme of a desulfurization series for ion exchanged Cu—Na—Y Zeolite. Each arrow shows the sulfur content in JP-8 in ppm.

FIG. 4 shows a scheme of a desulfurization series for Cu— and Co—Na—Y Zeolites. Each arrow shows the sulfur content in JP-8 in ppm.

FIG. 5 shows a scheme of a desulfurization series for Cu— and Ni—Na—Y Zeolites. Each number represents the sulfur content in the motor and aviation fuel in ppm.

FIG. 6 shows a scheme of a desulfurization series for Cu ion-exchanged Na—Y Zeolite and Ag—Cu wet impregnated Na—Y Zeolite. Each number represents the sulfur content in the motor and aviation fuel in ppm.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Hetero-atom containing compounds in fuels cause emissions of toxic air pollutants, such as SOx and NOx. These gases are the main source of particulates or soot, which significantly contribute to numerous problems, such as pollution, corrosion, health-related problems, and acid rain. In particular, nitrogen and sulfur organo-compounds in hydrocarbons have the ability to cause corrosion to the apparatus of a system, to decrease fuel quality, and to release NOx and SOx emissions, which will directly affect the environment (e.g., acid rains) and human health, and thereby prevent their use in fuel cells.

For instance, the aforementioned emissions contribute to adverse respiratory and cardiovascular effects. In particular, the emissions can cause cancer and several other health problems, including increased risk of premature mortality [1, 2]. Therefore, in order to lower the quantity of particulates emitted to the atmosphere, severe environmental regulations for commercial fuels have been implemented. The main objective of this measure is to improve the quality of air and prevent natural and health issues previously mentioned [3].

The presence of sulfur and nitrogen compounds in fuels also affects the performance of fuel based systems, such as automobiles, airplanes, and ships. For example, the emissions control system in vehicles, which is capable of oxidizing volatile organic matter and carbon monoxide, has its oxidation catalyst poisoned by the excess of sulfuric compounds [4]. In addition, sulfur and nitrogen might affect fuel quality and cause engine corrosion [5]. As nitrogen has more capability to be adsorbed if compared to sulfur, in order to perform desulfurization effectively, denitrogenation should be previously done [6-8].

Ultra-low sulfur fuels (e.g., with total sulfur of less than 1 ppm) are required for fuel cells because the sulfur contained in fuels causes the deactivation of the catalysts in both the reformer and the fuel cell electrodes [9, 10]. Sulfur can limit the usage of fuel cells for fixed and portable Auxiliary Power Units (APUs) applications. Jet fuels are particularly attractive for this application since they have high energy density and are readily available with little storage limitations [11]. This application remains problematic since commercial jet fuels can have up to 3000 ppmw of sulfur, as regulated by the U.S. Environmental Protection Agency (2010).

To meet the aforementioned technical and regulatory requirements, fuels must be submitted to ultrahigh desulfurization processes. The traditional hydrodesulfurization method (HDS) consists of a heterogeneous hydrogenation reaction, which requires hydrogen gas and severe operating conditions (40-50 bar and 300-400° C.) [12]. In order to avoid such conditions, oxidative desulfurization, microbes metabolism of sulfur compounds and selective adsorption have been proposed as alternatives to HDS [13,14]. However, oxidative desulfurization conventionally requires the usage of hydrogen peroxide, which is not readily available.

Moreover, metabolism of sulfur heterocycles by microbes is slow. In addition, the metabolism requires large holding tanks, and clean conditions. Such requirements can have a loss of desired components and be expensive. As a matter of illustration, benzene carbon-carbon bonds can be broken by enzymatic attacks [13].

Studies reveal that selective adsorption is, however, capable of removing the main sulfur and nitrogen compounds, such as sulfides, disulfides, thiols, thiophenes, benzothiophenes, anilines, pyrroles, indoles and carbazoles from fuels at ambient temperature and pressure [5-7, 15-17]. Selective adsorption is considered to be the most effective way of removing sulfur and nitrogen compounds to very low sulfur levels (<1 ppm) at very mild conditions.

State of Art

U.S. Pat. No. 4,634,515 discloses a sulfur trap for removing sulfur compounds (mercaptans, thiophene, and hydrogen sulfide) from a hydrofiner stream containing 1-50 ppm of sulfur that is supposed to be placed before a reformer unit with a sulfur sensitive catalyst. The sulfur trap comprises a bed of alumina supported nickel adsorbent of large crystallite size that contains over 50% of reduced nickel ions. The temperature for the desulfurization process ranges from 150° C. to 260° C. The patent claims that the average size of the sulfur adsorbent crystallite should be between 92 and 500 Å. It was found that a bigger average size of the crystallites enhances sulfur removal [18]. This process differs from Applicants' processes disclosed herein at least because the feed stream has low sulfur content, and the sulfur compounds removed by this process include mercaptans, thiophenes, and hydrogen sulfides. In contrast, Applicants' processes in some embodiments can remove these compounds in addition to DBT and DMDBT, which are very difficult to remove. Moreover, Applicants' processes in some embodiments can remove hetero-atom containing compounds at temperatures lower than 150° C. by using adsorbents other than nickel-based materials.

U.S. Pat. No. 5,993,516 describes an adsorbent for removing nitrogen from a feed gas of one or more gases with molecular dimensions equal or larger than methane. It is claimed that the adsorbent should be a zeolite clinoptilollite containing at least 17% and up to 95% of sodium ion exchangeable cations and at least one non-univalent cation, such as H⁺, NH₄⁺, K⁺, Li⁺, Rb⁺, and Ce⁺ [19]. This process differs from Applicants' processes disclosed herein at least because the stream has to be exclusively gaseous and it must have a specific molecular size. In addition, only specific types of adsorbents are used. Moreover, a process temperature range is not claimed. The aforementioned patent only discloses a clinoptilollite zeolite as the support for the ions, and there must be sodium exchangeable cations in addition to other ions in their material. However, Applicants' processes do not require the aforementioned constraints.

U.S. Pat. No. 5,919,354 describes a process for removing sulfur compounds from refinery feed stocks (preferably crude oils), refinery intermediates, refinery products (preferably liquid hydrocarbon fuels with carbon numbers ranging between 5 and 20), and mixtures thereof. The sorbents used in the process include natural or synthetic metal-exchanged Y-zeolites, which can be mixed with an inert material. The desulfurization is carried out at a temperature that ranges from ambient to reflux temperature, where pressures should not be greater than 698 kPa. The examples provided reached up to 52.1% sulfur reduction for an inlet concentration of 14,200 ppm at 251° C. and atmospheric pressure [20]. This process differs from Applicants' processes disclosed herein because it uses high pressure (up to 698 kPa). Moreover, the sulfur removal yield reached is low (maximum of 52%). Additionally, the aforementioned patent specifies the inlet stream as refinery feedstock, intermediates, or products and materials based on zeolite Y only. In contrast, Applicants' processes in some embodiments can remove hetero-atom from other hydrocarbon fluids and can use other materials for such processes.

U.S. International Patent Application No. WO 2003/068892 presents a process to reduce the sulfur content in transportation fuels to an ultra-low sulfur level (range not defined). The materials and methods described are applicable for motor vehicles and fuel cells and can be operated at ambient conditions or elevated temperatures and pressure. The claims filed consist of a desulfurization process comprising contacting the fuel with the selected adsorbent at a temperature within the range of 10 to 340° C. The adsorbent material might consist of a metal ion-exchanged zeolite, metal ion impregnated zeolite, transition metal chlorides, sulfide Co—Mo/alumina, and Ni based adsorbents. The transportation fuels included in the patent claims consist of naphtha, gasoline, model gasoline, diesel fuel, model diesel fuel, jet fuel, model jet fuel, and kerosene [21]. This process differs from Applicants' processes disclosed herein at least because the stream can only be a transportation fuel. In addition, only one adsorbent is used in a one-step approach. Furthermore, the aforementioned patent application does not disclose the amount of sulfur in the feed, which can be significant for a desulfurization process.

U.S. Patent Application No. US 2004/0118747 describes a process for removing sulfur compounds from fuels comprising a monolithic sulfur-adsorbent reactor. The structure of the beds consists of honeycomb shaped packings with internal voids. The claims contained in the present patent include the addition of metal active sites in the void space channels of the monolithic reactor that is operated in a temperature range of 25° C. to 400° C. The adsorbents can be selected from Co, Ni, Mo, Cu, Cr, W, Mn, Fe, Zn oxides or active metals supported on carbon or zeolite [22]. This process differs from Applicants' processes disclosed herein at least because it is not a selective multi-step approach and the stream is limited to fuels only. In addition, the aforementioned patent application only discloses the use of a monolithic reactor, whereas the Applicants' processes can utilize other reactors.

U.S. International Patent Application No. WO 2005/075608 discloses a method for deep denitrogenation of hydrocarbon fuels by contacting the fuel with an adsorbent that preferentially adsorbs organo-nitrogen compounds comprising anilines, pyrroles, indoles, and carbazoles. The adsorbent can contain a metal or a metal cation that is able to complex with the compounds to be removed. The claimed metals are Mn²⁺, Fe²⁺, Co²⁺, Ni²⁺, Cu⁺, Zn²⁺, Ga³⁺, Pd⁰, Ag⁺, and Cd²⁺ ion exchanged in zeolite (X, Y, LSX, MCM-41 and mixtures thereof). It is claimed that the adsorption should be performed in a specific temperature and pressure (not specified), and a change in the conditions could be able to release the organo-nitrogen compounds from the adsorbent [23]. This process differs from Applicants' processes disclosed herein at least because it is limited to nitrogen compounds. Moreover, the temperature range is not specified and the process is not multi-step. Additionally, the process in the aforementioned patent application is limited to hydrocarbon fuels only.

European Patent No. EP 1 550 505 discloses an adsorbent capable of removing a variety of sulfur compounds from hydrocarbon fuels. The process presented by the patent can produce a stream with low sulfur concentration (<0.1 ppm) at ambient conditions by contacting the fuel with an adsorbent. The adsorbents claimed include Ce based adsorbents supported on oxides or zeolites. Furthermore, there is a second stage of the process, which comprises contacting the desulfurized fuel with a partial-oxidation reforming catalyst at temperatures under 200° C. to produce hydrogen for fuel cell applications [24]. This process differs from Applicants' processes disclosed herein at least because the solid material is limited to Ce-based adsorbents, and the stream is limited to hydrocarbon fuels. In addition, the aforementioned patent can only remove sulfur compounds under ambient conditions.

U.S. Patent Application No. 2005/0263441 describes a process for removing contaminants comprising nitrogen and sulfur compounds from liquid hydrocarbon fuels using a nanostructured material as adsorbent at ambient conditions. The adsorbent comprises a nanostructured JT phase titanium oxide TiO₂-x (where 0≤x≤1) having a thermally stable orthorhombic crystalline structure composed of overlapped semitubes. The adsorbent can also contain a transition metal oxide promoter. The combustibles mentioned in the aforementioned patent application are gasoline, diesel, kerosene, straight run gas oil, and heavier fractions. It was claimed that the process consists of contacting the adsorbent with the liquid hydrocarbon fuel [25]. This process differs from Applicants' processes disclosed herein at least because the stream is limited to hydrocarbon fuels and the removal of sulfur components occurs only under ambient temperature.

U.S. Pat. No. 7,094,333 describes a method for removing thiophene and thiophenic compounds from liquid fuel by contacting the liquid fuel with an adsorbent that preferentially adsorbs those organosulfur compounds by π-complexation bonds. The patent claimed that the adsorbents include Mn²⁺, Fe²⁺, Co²⁺, Ni²⁺, Cu²⁺, Zn²⁺, Ga³⁺, Pd⁰, or Ag⁺ ion exchanged zeolites and the adsorption happens at a selected temperature and pressure (values not given in the patent). The dehydrated adsorbents can be regenerated by applying a change in the operation conditions. Furthermore, the method described is capable of removing aromatic compounds, but it is slightly more selective to remove thiophenic compounds [26]. This process differs from Applicants' processes disclosed herein at least because it removes only thiophenic compounds from liquid fuels. The sulfur removal occurs in one single step, and the reaction conditions are not specified. Additionally, the aforementioned patent discloses its materials as zeolite based-adsorbents only.

U.S. Pat. No. 7,704,383 discloses a mobile fuel filter for desulfurization of diesel fuel (post-refinery). The filter contains an adsorbent that comprises one or more inorganic oxides, such as alumina, kaolinite, zeolite, super acid, titania, and silicon dioxide. The feed contains substituted alkylthiophenes, benzothiophenes, and dibenzothiophenes. Sulfur content of two different feeds is less than 100 ppm or below 2000 ppm. It is claimed that the fuel filter removed benzothiophenes and dibenzothiophenes and their derivatives. The fuel filter reduces the sulfur level to 15 ppm or less [27]. However, the aforementioned patent differs from Applicants' processes disclosed herein at least because it does not disclose the temperature of the operation, which can be a significant parameter during desulfurization processes. The aforementioned patent also differs from Applicants' processes at least because the inlet stream for the process only includes diesel fuel and the sulfur range for this inlet stream is limited to up to 2,000 ppm of sulfur.

U.S. Pat. No. 8,021,540 discloses a method for desulfurizing a kerosene or gas oil containing thiophenes, benzothiophenes, and dibenzothiophenes by contacting the fuel with an adsorbent. It was claimed that the adsorbents comprise proton-type faujasite zeolites, proton-type mordenites and proton-type beta-zeolites. The kerosene or gas oil was desulfurized to 1 ppm or less from a feed containing over 80 ppm of sulfur. There is no claim for the temperature of the process [28]. The aforementioned process differs from Applicants' processes disclosed herein at least because the fuel is limited to kerosene and gas oil. Additionally, the solid nanomaterial is limited to faujasite, modernites and beta zeolites and the inlet stream has low sulfur content.

U.S. Patent Application Publication No. 2011/0138781 discloses a process for removing sulfur from hydrocarbon fuels for onboard vehicle applications by contacting the fuel with copper-1,3,5-benzenetricarboxylic acid Metal-Organic Framework (MOF). The inlet stream is typically low-sulfur content diesel fuel (8 to 15 ppm). It is claimed that the process is suitable for all commercial fuels. A fixed bed reactor is used for continuous flow and to minimize the number of containers and apparatus. The reaction preferably takes place at 0-100° C., and 0.5 to 5 bar during 5-60 minutes [29]. This process differs from Applicants' processes disclosed herein at least because the stream is limited to hydrocarbon fuels for fuel cell applications. In addition, the solid nanomaterial is restricted to being 1,3,5-benzenetricarboxylic acid Metal-Organic Framework (MOF). Moreover, the inlet stream contains very low sulfur concentration. The conditions under which the removal of the sulfur compounds occur also differ from Applicants' processes.

U.S. Pat. No. 8,142,647 discloses a process for removing aromatic sulfur compounds (benzothiophene and dibenzothiophene) from a C₆-C₂₀ aromatic and/or aliphatic stream, where the adsorbent comprises 2,4,5,7-terenitro-9-fluorenylideneaminooxy propionic acid (TAPA) functionalized silica. The inventors studied the competitive adsorption and found that when toluene is added to n-heptane (50-50 vol %), it decreases the binding capacity of DMDBT. The adsorbent is regenerated at about 50-100° C. [30]. This process differs from Applicants' processes disclosed herein at least because the compounds to be removed are limited to thiophenes and dibenzothiophenes and the inlet stream is limited to a range of hydrocarbons. In addition, the material used to remove those compounds can only be TAPA functionalized silica. Moreover, the conditions under which the removal of sulfur compounds occurs are not specified.

U.S. Pat. No. 8,323,603 discloses a desulfurization system for producing a hydrocarbon fuel stream with less than 50 ppb of sulfur from a gas-phase fuel. The fuel described herein typically ranges from 1 to 500 ppm of sulfur (particularly natural gas, propane or liquefied petroleum gas). The main sulfur compounds present in such fuels are carbonyl sulfide, hydrogen sulfide, thiophenes, mercaptans, and sulfoxides. The described desulfurization process can be utilized without hydrogen at temperatures lower than 100° C. for fuel cell applications. It was claimed that there is a process comprising a sequential sulfur adsorbent system capable of removing sulfur from such fuels at those conditions. The system sequence was claimed as a copper exchanged zeolite Y adsorbent, a hydrated alumina adsorbent, and finally a selective sulfur adsorbent, which may be selected from a variety of adsorbents (Cu, Ag, Mn, Fe, Ca, Ce, La, Sr, Pr, and Nd ion exchanged Y zeolites) according to the affinity with the sulfur compounds in the stream [31]. This process differs from Applicants' processes disclosed herein at least because the inlet stream is a gas-phase fuel with up to 500 ppm of sulfur. Moreover, the temperature range of operation is under 100° C.

U.S. International Patent Application No. WO 2013/043629 discloses a method for removing sulfur compounds from low sulfur hydrocarbon fuels containing 100 ppm of sulfur or less. The method consists of contacting the fuel with a sulfur sorbent material synthesized with active copper components on zeolite (i.e., framework types of AFS, ATS, BEA, BOG, CON, DFO, EMT, EON, ETR, EZT, FAU, a structural EMT-FAU intermediate, GME, LTL, MAZ, MFJ, MOR, MOZ, MSE, OFF, SAO, SFO, and/or UFI, or a combination or structural intermediate thereof) or the following mesoporous supports: MAPO-46, MAPO-36, SSZ-55, zeolite beta, boggsite, CIT-1, SSZ-26, SSZ-33, DAF-1, EMC-2, ECR-1, TNU-7, ECR-34, EMM-3, zeolite Y, zeolite X, SAPO-37, CSZ-1, ECR-30, ZSM-20, ZSM-3, gmelinite, zeolite L, perlialite, LZ-212, mazzite, LZ-202, omega, ZSM-4, ZSM-18, ECR-40, mordenite, ZSM-10, MCM-68, offretite, LZ-217, STA-1, SSZ-51, UZM-5, MCM-41, or SBA-15. The process operates at 200° C. or less and the fuel product sulfur content is decreased by at least 20%. It is claimed in this patent that the fuels in the feed stream include naphta, gasoline, diesel, jet fuel, and kerosene streams or a combination thereof [32]. This process differs from Applicants' processes disclosed herein at least because the inlet stream contains only up to 100 ppm sulfur and the sulfur removal occurs in a one-step process.

In addition to the previously mentioned patents, several research articles report on desulfurization via adsorption. Most of these articles discuss desulfurization at room temperature with a few exceptions at higher temperatures.

In most of the existing research works, the fluid includes a model fuel containing a solution of hexane/heptane/octane and one thiophenic sulfur compound only. However, real fuel contains hundreds of hydrocarbons (e.g., alkanes, alkenes, aromatics, etc.) and different types of sulfur compounds (e.g., sulfides, disulfides, thiols, thiophenes, benzothiophenes, and dibenzothiophenes). Moreover, compounds such as aromatics can adsorb on to the active sites and consequently reduce desulfurization. Therefore, despite being useful for fundamental studies, model fuels are not truly representative of real fuels and only a few have been discussed here for desulfurization applications.

A wide range of sulfur concentrations in the feed for adsorptive desulfurization can be found in the literature. Several researchers used feed containing sulfur below 400 ppm [33-35]. In that case, sulfur levels were reduced to below 1 ppm. On the other hand, higher sulfur concentrations in the inlet, ranging from 1000 to 1675 ppm, have also been reported [36-40]. However, when the feed contained higher sulfur level, the processes were only able to reduce sulfur levels to the range of 10 to 30 ppm [36-40].

Cu, Ni, and Ag ion-exchanged Y zeolites have been commonly used adsorbents and found to be very effective for removing difficult to remove sulfur compounds at room temperature as well as at higher temperatures [33-35, 39, 41]. Other adsorbent materials include Ni/SiO₂—Al₂O₃ [36] and Ni—Ce/Al₂O₃—SiO₂ [37]. Adsorptive desulfurization has been reported at temperatures as high as 220° C. [36].

Pretreatments have largely been used in order to enhance sulfur adsorption. Velu et al. [36] fractionated JP-8 (736 ppm sulfur) to light fraction JP-8 (380 ppm sulfur). They mentioned that, for easy integration of desulfurization unit with reformer of SOFC, desulfurization needs to be carried out at around 200-220° C. Fractionation mainly removed C3-BT, especially 2,3,7-trimethylbenzothiophene (2,3,7-TMBT). Their process reduced sulfur level below 30 ppm.

Wang et al. reported pervaporation to reduce sulfur in feed from 1675 ppm to 290 ppm and then conduct desulfurization at room temperature. The sulfur content was reduced to less than 10 ppm. They used Cu—Y Zeolite and some other adsorbents that were not disclosed in the article. Pervaporation is a process for extracting aromatics from aliphatic hydrocarbons by solvent diffusion transport through a membrane [38].

Jhung and Ahmed performed adsorptive denitrogenation with CuCl₂/Mi(Cr) wet impregnated on model fuels-solutions of 10,000 ppm of benzothiophenes, quinolone, and indoline. The solutions were separately dissolved in a mixture of 25% p-xylene and 75% n-octane. These three solutions were then diluted and mixed to form a model fuel with various different concentrations. The adsorption reactions were carried out at ambient temperature in a batch reactor with stirrer. It was reported that the N compounds were adsorbed to a greater extent than benzothiophenes for a solution of 400 ppm quinolone, 400 ppm indoline, and 800 ppm benzothiophene [6].

Nikou et al. reported desulfurization and denitrogenation of a set of model diesel fuels using the aluminosilicate mesostructured MSU-S modified with phosphotungsten acid (HPW) and nickel-oxide-HPW (NiOHPW). Three different model fuels were used, including a nitrogen rich model diesel fuel (containing carbazole and quinolone—269 ppm N), a sulfur rich fuel (containing thiophene and dibenzothiophene—1221 ppm S) and a sulfur-nitrogen rich fuel (containing all the compounds previously mentioned—271 ppm N, 1242 ppm S). The solvents used were n-hexadecane and n-octane, and the solution contained aromatics, such as naphthalene and toluene. The experiments were carried in a batch reactor with stirrer at room temperature. It was found that both adsorbents show selective adsorption towards nitrogen over sulfur compounds [7].

Heteroatom Removal Processes

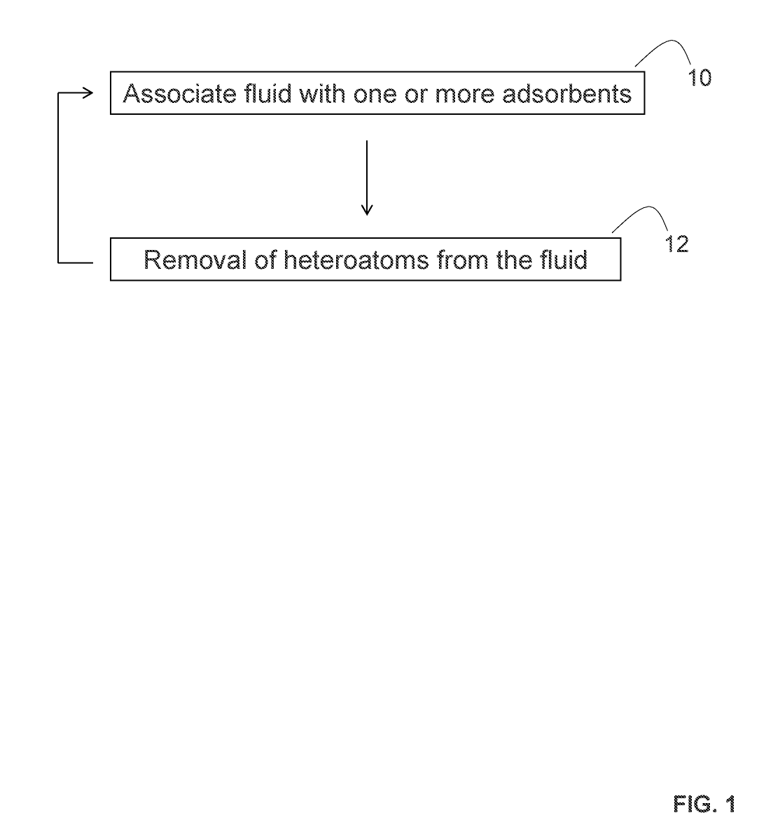

In some embodiments, the present disclosure provides highly efficient methods for removing heteroatoms (e.g., sulfur and nitrogen heteroatoms) from a fluid (e.g., hydrocarbons) under mild conditions. In some embodiments illustrated in FIG. 1, the methods of the present disclosure involve a step associating a fluid with one or more adsorbents (step 10) to result in the removal of the heteroatoms from the fluids (step 12). In some embodiments, the methods of the present disclosure include the sequential association of the fluid with adsorbents (e.g., compound-specific solid nanomaterials). In some embodiments, the present disclosure includes specific compositions of matter, that when used in a selected serial manner, provide synergy and a more economical removal of the heteroatoms from a fluid as the basis for a new process.

Heteroatoms

The methods of the present disclosure may be utilized to remove various types of heteroatoms from fluids. In some embodiments, the heteroatoms may be in individual form. In some embodiments, the heteroatoms may be associated with a compound (i.e., a heteroatom-containing compound). In some embodiments, the heteroatoms include, without limitation, sulfur-containing compounds, nitrogen-containing compounds, oxygen-containing compounds, and combinations thereof. In some embodiments, the sulfur-containing compounds include, without limitation, sulfides, disulfides, thiols, mercaptans, hydrogen sulfides, thiophenes, benzothiophenes, dibenzothiophenes, and combinations thereof. In some embodiments, the nitrogen-containing compounds include, without limitation, anilines, pyrroles, indoles, carbazoles, and combinations thereof. In some embodiments, the oxygen-containing compounds include, without limitation, phenols, alcohols, acids and combinations thereof.

Fluids

The methods of the present disclosure can be utilized to remove heteroatoms from various fluids. In some embodiments, the fluids include, without limitation, fuels (e.g., jet fuels), hydrocarbons (e.g., neat hydrocarbons), alcohols, water, organic solvents, and combinations thereof. In some embodiments, the methods of the present disclosure can remove various heteroatoms (e.g., sulfur) from the aforementioned fluids more efficiently than existing processes.

The fluids of the present disclosure may be in various states. For instance, in some embodiments, the fluids of the present disclosure are in a state that include, without limitation, a gaseous state, a liquid state, and combinations thereof. In some embodiments, the fluids of the present disclosure are in a liquid state. In some embodiments, the fluids of the present disclosure are in a gaseous state.

In some embodiments, the fluids of the present disclosure include a hydrocarbon fine chemical. In some embodiments, the fluids of the present disclosure include a hydrocarbon fuel. In some embodiments, the hydrocarbon fuel is a liquid. In some embodiments, the hydrocarbon fuel is a gas. In some embodiments, the hydrocarbon fuel includes, without limitation, diesel fuel, kerosene, gasoline, natural gas, and combinations thereof.

The fluids of the present disclosure can include various amounts of heteroatom content. For instance, in some embodiments, the fluids of the present disclosure include heteroatom contents that range from about 100 parts per million by weight (ppmw) to about 5,000 ppmw. In some embodiments, the fluids of the present disclosure include heteroatom contents that range from about 1 ppmw to about 5,000 ppmw. In some embodiments, the fluids of the present disclosure include heteroatom contents of more than about 100 ppmw. In some embodiments, the fluids of the present disclosure include heteroatom contents of more than about 500 ppmw. In some embodiments, the fluids of the present disclosure include heteroatom contents of more than about 1,000 ppmw. In some embodiments, the fluids of the present disclosure include heteroatom contents of more than about 1,500 ppmw. In some embodiments, the fluids of the present disclosure include heteroatom contents of more than about 2,000 ppmw.

In some embodiments, the fluids of the present disclosure include heteroatom contents of more than about 3,000 ppmw. In some embodiments, the fluids of the present disclosure have a total sulfur content of about 3,000 ppmw or greater. In some embodiments, the fluids of the present disclosure have a total nitrogen content of about 500 ppmw or greater. In some embodiments, the fluids of the present disclosure have a total nitrogen content of about 10 ppmw or greater.

Adsorbents

The methods of the present disclosure may utilize various types of adsorbents (adsorbents are also referred to herein as catalysts or solid nanomaterials). In some embodiments, the one or more adsorbents are the same. In some embodiments, the one or more adsorbents are different. In some embodiments, the one or more adsorbents include, without limitation, activated carbon, zeolites, ion exchanged zeolites, ion impregnated zeolites, alumina, alumina nanowires, carbon-based supports, and combinations thereof.

In some embodiments, the adsorbents of the present disclosure include one or more adsorbent components. In some embodiments, the adsorbent components of the present disclosure include, without limitation, active metals, transition metals, oxides thereof, sulfides thereof, and combinations thereof.

In some embodiments, the adsorbents of the present disclosure include transition metals. In some embodiments, the transition metals include, without limitation, Co, Cu, Ce, Ni, Fe, Mn, Pd, Ag, W, Zn, Pt, Au, Cr, V, Ti, Mo, oxides thereof, sulfides thereof, and combinations thereof. In some embodiments, the transition metals are supported on various supports, such as alumina or alumina nanowires.

In some embodiments, the adsorbents of the present disclosure may include a single transition metal. In some embodiments, the adsorbents of the present disclosure can include a plurality of transition metals. In some embodiments, the adsorbents of the present disclosure can include two or more transition metals. In some embodiments, the adsorbents of the present disclosure can include a plurality of different transition metals (e.g., one, two, three or more transition metals at the same time). In some embodiments, the adsorbents of the present disclosure may include bi-metallic materials, tri-metallic materials, and combinations thereof.

In some embodiments, the adsorbents of the present disclosure include zeolites. In some embodiments, the zeolites can include, without limitation, X, Y, Beta, Mordenite, and ZSM-5 zeolites. In some embodiments, the zeolites may be associated with cations. In some embodiments, the cations include, without limitation, Na, H, K, and combinations thereof.

In some embodiments, the adsorbents of the present disclosure include H or metals. In some embodiments, the metals include, without limitation, Na, K, and combinations thereof.

In some embodiments, the adsorbents of the present disclosure are affixed to a solid support. In some embodiments, the solid support includes, without limitation, alumina, alumina nanowires, activated carbon, zeolites, and combinations thereof.

In some embodiments, the adsorbents of the present disclosure include ion exchanged zeolites. In some embodiments, the adsorbents of the present disclosure include ion impregnated zeolites. In some embodiments, the adsorbents of the present disclosure are wet impregnated.

The adsorbents of the present disclosure can have various surface areas. For instance, in some embodiments, the adsorbents of the present disclosure have a surface area of at least 50 m²/g. In some embodiments, the adsorbents of the present disclosure have a surface area of at least 100 m²/g. In some embodiments, the adsorbents of the present disclosure have a surface area ranging from about 150 m²/g. In some embodiments, the adsorbents of the present disclosure have a surface area ranging from about 100 m²/g to about 1000 m²/g. In some embodiments, the adsorbents of the present disclosure have a surface area ranging from about 150 m²/g to about 600 m²/g.

The adsorbents of the present disclosure can be fabricated by various methods. Such methods can include ion exchanged or wet impregnated techniques outlined in Example 1. In some embodiments, the adsorbents of the present disclosure include a single layer. In some embodiments, the adsorbents of the present disclosure include multiple layers. In some embodiments, adsorbent stacking order may be arranged to remove specified heteroatom containing molecules.

In some embodiments, the adsorbents of the present disclosure are stored in a proper container so that the adsorbents remain non-oxidized and preserved in order to maintain their active sites during transportation, storage and reactor loading. In some embodiments, adsorbents are activated prior to heteroatom association (e.g., sulfur adsorption) inside a unit (e.g., a tubular furnace).

Association of Adsorbents with Fluids

Various methods may be utilized to associate adsorbents with fluids. For instance, in some embodiments, the associating occurs by contacting the fluid with one or more adsorbents. In some embodiments, the associating occurs by associating the fluid with a single adsorbent.

In some embodiments, the associating occurs in a single step. In some embodiments, the associating occurs in multiple steps. In some embodiments, the associating occurs by associating the fluid with a plurality of adsorbents in a sequential manner. In some embodiments, the sequential association is arranged to maximize heteroatom removal. In some embodiments, the heteroatom removal efficacy is maximized by requiring less adsorbents, requiring less processing time, enhancing heteroatom removal efficiency, removing more heteroatoms, or combinations thereof. In some embodiments, the adsorbents are sequenced in a specific order to selectively remove competing heteroatoms and fluid components.

In some embodiments, the associating occurs without any fluid pre-treatment steps. For instance, in some embodiments, the associating occurs without any fluid fractionation, prevaporation, dissolution, or dilution steps.

Heteroatom Removal

The methods of the present disclosure can occur under various reaction conditions. For instance, in some embodiments, heteroatom removal can occur at temperatures ranging from about 10° C. to about 500° C. In some embodiments, heteroatom removal can occur at temperatures ranging from about 25° C. to about 250° C. In some embodiments, heteroatom removal can occur at temperatures ranging from about 100° C. to about 250° C. In some embodiments, heteroatom removal can occur at temperatures ranging from about 150° C. to about 500° C. In some embodiments, heteroatom removal can occur at temperatures of more than about 100° C.

In some embodiments, the methods of the present disclosure may be utilized to remove different heteroatoms (e.g., heteroatom containing compounds) simultaneously. In some embodiments, the methods of the present disclosure involve contacting the adsorbent with the fluid in a specific order. In some embodiments, a specific order of adsorbents effectively removes more than one type of heteroatoms employing the same series. In some embodiments, more than one adsorbent may be utilized in the same step or in sequential steps. In some embodiments, the utilization of multiple adsorbents improves heteroatom adsorption efficiency and selectivity.

In some embodiments, heteroatom removal occurs without the addition or utilization of any non-oxygen gases. In some embodiments, heteroatom removal occurs without the addition or utilization of reactive gases. In some embodiments, heteroatom removal occurs without the addition or utilization of H₂.

In some embodiments, heteroatom removal reduces the heteroatom content of the fluid to below 30 ppmw. In some embodiments, heteroatom removal reduces the heteroatom content of the fluid to below 10 ppmw. In some embodiments, heteroatom removal reduces the heteroatom content of the fluid to below 1 ppmw.

In some embodiments, heteroatom removal results in a reduction of heteroatoms in the fluid by more than about 50%. In some embodiments, heteroatom removal results in a reduction of heteroatoms in the fluid by more than about 80%. In some embodiments, heteroatom removal results in a reduction of heteroatoms in the fluid by more than about 85%. In some embodiments, heteroatom removal results in a reduction of heteroatoms in the fluid by more than about 90%. In some embodiments, heteroatom removal results in a reduction of heteroatoms in the fluid by more than about 99%. In some embodiments, heteroatom removal results in a reduction of heteroatoms in the fluid by more than about 99.6%.

In some embodiments, heteroatoms are removed from chemical components contained in the fluid. In some embodiments, heteroatom removal occurs non-selectively. In some embodiments, heteroatoms are removed simultaneously. In some embodiments, heteroatom removal occurs selectively.

Applications

In some embodiments, the methods of the present disclosure may be applied to systems where removal of heteroatom-containing compounds from a fluid (e.g., fluids containing hydrocarbons) is required, but severe conditions such as extremely high temperatures and pressures and reactive gas cannot be used or when reaching those conditions is not feasible economically. Furthermore, the absence of such harsh conditions, particularly reactive gas circulation, makes the system easier to engineer and is therefore attractive both economically and environmentally. In some embodiments, the methods of the present disclosure selectively remove heteroatom containing molecules, such as sulfur containing organics, from hydrocarbons.

In some embodiments, the methods of the present disclosure can be used to remove sulfur and nitrogen heteroatoms from a fluid (e.g., hydrocarbons or fuels) with total sulfur content of 3000 ppmw or greater (including sulfides, thiophenes, benzothiophenes, and dibenzothiophenes) and total nitrogen content of 500 ppmw or greater (including anilines, pyrroles, indoles, and carbazoles) at mild conditions by contacting the fluid with a series of adsorbents that might or might not be the same depending on the chemical composition of the compounds to be removed.

In some embodiments, the method of the present disclosure can be utilized to remove over 99% of sulfur from various fluids (e.g., logistical JP-8 fuel, a light kerosene that the US military uses in all its vehicles and jets).

In some embodiments, the methods of the present disclosure can be utilized for one of more of the following applications: (1) removal of sulfur compounds from liquid fuels, such as jet fuel, kerosene, diesel, gasoline, and combinations thereof; (2) removal of nitrogen compounds from liquid fuels, such as jet fuel, kerosene, diesel, gasoline; (3) removal of sulfur compounds from gaseous fuels, such as natural gas, exhaust gases from ships, and power plants; (4) removal of nitrogen compounds from gaseous fuels, such as natural gas, exhaust gases from ships, and power plants; (5) desulfurization of hydrocarbons for reformers, such as onboard fuel cells in cars and aircrafts, or for auxiliary power units (APU); and (6) selective removal of unwanted metal ions, residual organic solvents, sulfur or other heteroatom containing compounds from drugs, foods, cosmetics, water and combinations thereof. In some embodiments, the methods of the present disclosure can be utilized in conjunction with the current hydrodesulfurization (HDS) method implemented in refineries to produce cleaner fuels.

Advantages

In some embodiments, the methods of the present disclosure are better than existing methods because they do not require severe operating conditions and reactive gases, such as H₂. Especially when compared to low temperature (e.g., 25-200° C.) adsorption processes, the methods of the present disclosure promote highly selective heteroatom adsorption using a multistep approach.

Previous methods show adsorptive desulfurization using one type of material. However, in the methods of the present disclosure, it was found that different adsorbents have varying affinity towards different sulfur and nitrogen compounds and that there is a certain sequence for placing the materials in order to perform heteroatom removal efficiently and selectively.

Another advantage of the methods of the present disclosure is enhanced sulfur removal with higher sulfur adsorption capacity because the process involves step-wise sulfur reduction using one or more adsorbents. Most of the existing methods make use of one single adsorbent for the entire desulfurization unit. By setting a sequence of different adsorbents, it is possible to selectively remove specific compounds at each step of the process and therefore increase its efficiency since different adsorbents have varying affinity towards different compounds.

Another advantage of the methods of the present disclosure is that the methods of the present disclosure do not require the use of any reactive gas(es). Therefore, the methods of the present disclosure are potentially safer and less expensive than existing processes.

Applicants have discovered that the order in which the adsorbents are placed affects the efficiency of the heteroatom removal process. This is a significant finding, and to the best of Applicants' knowledge, it has not yet been reported in the literature. Moreover, this kind of methodology can be applied to other processes that involve catalysts or adsorbents. For instance, the methods of the present disclosure can be applied to purify water or exhaust gas from refinery, paper industry, and power plants where multiple impurities are contained.

A more specific advantage of the methods of the present disclosure is that enhanced sulfur removal with higher sulfur adsorption capacity can be attained because, in some embodiments, the methods of the present disclosure can involve step-wise sulfur reduction using one or more adsorbents.

### ADDITIONAL EMBODIMENTS

Reference will now be made to more specific embodiments of the present disclosure and experimental results that provide support for such embodiments. However, Applicants note that the disclosure herein is for illustrative purposes only and is not intended to limit the scope of the claimed subject matter in any way.

### Example 1. Synthesis and Use of Adsorbents for Desulfurization

In this example, Applicants demonstrate the synthesis and use of adsorbents for desulfurization reactions. Adsorbents are also referred to herein as catalysts. The catalysts are synthesized using ion exchanged or wet impregnated techniques.

### Example 1.1. Catalyst Synthesis by Ion Exchange Techniques

The list of catalysts synthesized for ion exchange techniques include Ag, Cu, Ni, Co, and Fe, and mixtures thereof exchanged in Na and H Y zeolites (aluminosilicate crystalline structure with large area that supports the active sites). Metal salts, such as nitrates, are used as precursors for the catalysts.

First, a nitrate precursor of the desired metal (Cu, Ag, Ni, Co, or Fe) is dissolved in deionized (DI) water at room temperature. Then, zeolite powder is added to the aqueous solution of the metal salt. Contact time is for 24 hours at room temperature. Thereafter, the solution is filtered and washed with large amounts of DI water to eliminate traces of metal that was not ion exchanged with the support. The material is then placed in the oven to dry at 105° C. (drying time: 9-24 hours). Activation of the catalyst and removal of moisture from the voids is carried out in a furnace at 450° C. for 8 hours in He/Air gas. The catalysts are then stored in a moisture free environment (desiccator) to avoid oxidation of active sites where desulfurization takes place.

### Example 1.2. Catalyst Synthesis by Wet Impregnation Techniques

The list of catalysts synthesized for this process includes Ag, Cu, Fe, Ni, Co, Fe, and mixtures thereof impregnated on Na and H Y Zeolites. Metal nitrates were used as precursors for the catalysts.

First, a nitrate precursor of the desired metal (Cu, Ag, Ni, Co, or Fe) is dissolved in DI water at room temperature. Thereafter, zeolite powder is added to the aqueous solution of metal salt, which is left stirring for 20 minutes at room temperature. The material is then placed in the oven to dry for 9 hours at 105° C. Activation of the catalyst and removal of moisture from the voids is carried in a furnace at 450° C. for 8 hours in He/Air gas. The catalysts are then stored in a moisture free environment (desiccator) to avoid oxidation of active sites where desulfurization takes place.

### Example 1.3. Additional Catalyst Synthesis Methods

Solid state ion exchange synthesis methodologies can also be utilized to synthesize catalysts. Such a method does not require water. For instance, an adsorbent (e.g., a zeolite) can be physically mixed with a metal salt and calcined in a temperature range of 300-550° C.

For catalyst synthesis, the ion exchange method can be chosen from different techniques, including solid, liquid or vapor ion exchange. Solid ion exchange has particular potential to decrease the catalyst production cost since it does not require the use of water for the salt solution. Therefore, the filtration step is also eliminated.

Industrial scale filtration processes require pumps and large facilities that result in elevated costs for industry. Moreover, by adopting a simpler ion exchange technique, it is possible to produce the same amount of catalyst in less time, when compared to liquid ion exchange.

In addition, it is possible to optimize the conditions of the catalyst synthesis stages, such as drainage and calcination time, temperature and environment. The loading of metal on the catalyst can also be adjusted in order to spend the optimum amount of metal salt and to perform desulfurization efficiently.

### Example 1.4. Sulfur Removal

Sulfur removal is done in a step-wise manner using batch reactors that mimic a flow reactor. The experiments were carried out in a temperature range of 100−250° C. The reactors are cooled down to around 33° C. and the contents are then vacuum filtered.

### Example 2. Multi-Step Desulfurization of Jet Fuel with Silver-Impregnated Na—Y Zeolite

In this Example, Applicants demonstrate the desulfurization of jet fuel by utilizing silver-impregnated Na—Y Zeolites as adsorbents. The zeolites were wet impregnated. The zeolites are also referred to as catalysts.

### Example 2.1. Catalyst Preparation

About 0.28 g of silver nitrate (AgNO₃) was added to 20 mL of DI water and stirred for 5 minutes until the salt completely dissolved. 5.82 g of Na—Y Zeolite was added to the solution and was left to stir for an additional 20 minutes. The solution was then placed in a Petri dish and dried at 105° C. for 24 hours. The catalyst was calcined at 450° C. for 3 hours in helium gas. The catalyst (referred to herein as 3% Ag/Na—Y Zeolite) was then ready to be used.

### Example 2.2. Sulfur Adsorption

22 mL of JP-8 containing 1,280 ppm of sulfur (sulfides, thiols, thiophenes, benzothiophenes and dibenzothiophenes) and 0.6 g of 3% Ag/Na—Y Zeolite were put in contact inside the reactor vessel. The reactor was properly sealed and placed in the oven (oven temperature=176° C.) for 3 hours. The reactor was cooled to a temperature around 33° C. and the mixture fuel-catalyst was vacuum filtered.

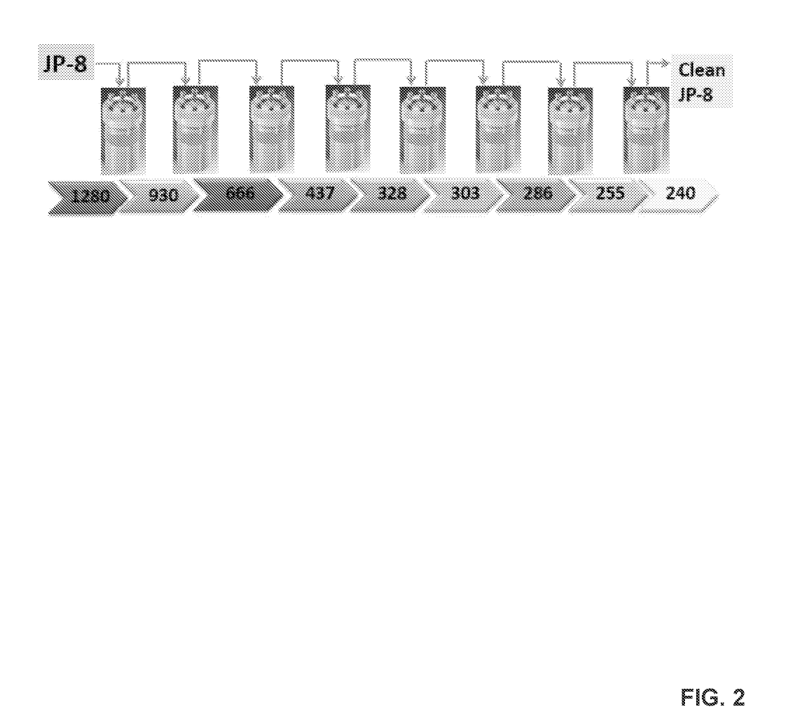

The same sulfur adsorption process was applied to the treated JP-8 fuel from the previous step. The second step desulfurization produced a fuel containing 666 ppm of sulfur. The procedure was then performed in a series of 8 sulfur adsorption steps (FIG. 2). The sulfur content in the inlet and outlet streams of each step is represented by the arrows. After 8 steps, the JP-8 had an overall 81% of sulfur removed using a total of 6.8 g of adsorbent.

### Example 3. Desulfurization of Jet Fuel with Copper Ion Exchanged in Na—Y Zeolite

In this Example, Applicants demonstrate the desulfurization of jet fuel by utilizing copper ion-exchanged Na—Y Zeolites (Cu—Na—Y Zeolite) as adsorbents. The zeolites were wet ion exchanged. The zeolites are also referred to as catalysts.

### Example 3.1. Catalyst Preparation

About 30 g of copper nitrate (Cu(NO₃)₂.H₂O) was added to 230 mL of DI water and stirred for 5 minutes until the salt completely dissolved. Next, 9 g of Na—Y Zeolite was added to the solution that was left to ion exchange stirring for 24 hours. The solution was washed, vacuum filtered, and placed in a Petri dish to dry at 105° C. for 9 hours. The catalyst was calcined at 450° C. for 8 hours. The catalyst (i.e., ion exchanged Cu—Na—Y Zeolite) was then ready to be used.

### Example 3.2. Sulfur Adsorption

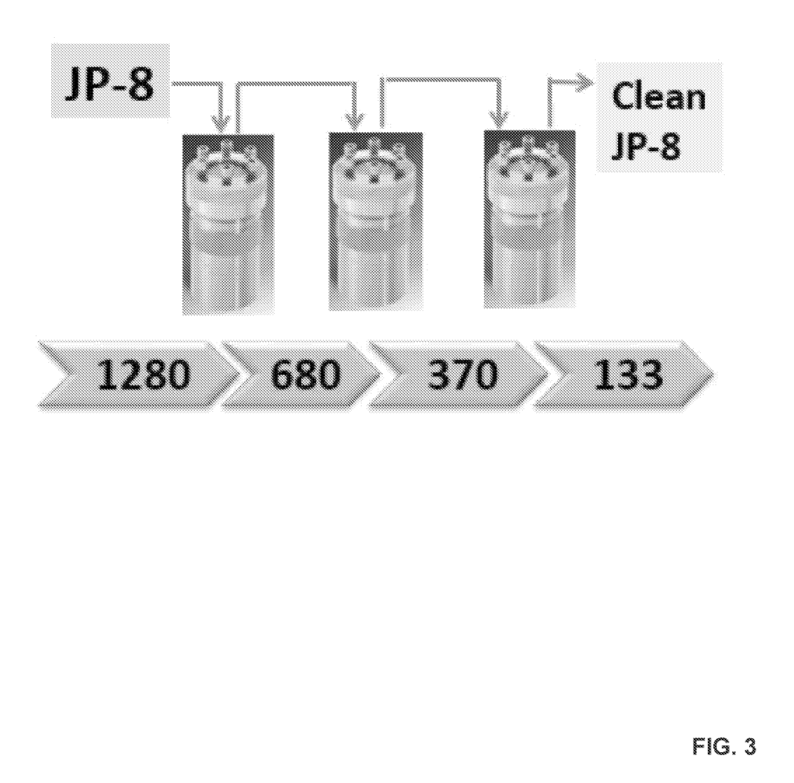

The series of Example 3 consists of a three step desulfurization using Cu—Na—Y Zeolite as a catalyst (FIG. 3). 27 mL of JP-8 containing 1,280 ppm of sulfur (sulfides, disulfides, thiols, thiophenes, benzothiophenes and dibenzothiophenes) and 0.74 g of Cu—Na—Y Zeolite were put in contact inside the reactor vessel. The reactor was properly sealed and placed in the oven (oven temperature=176° C.) mixture for 3 hours. The reactor was cooled to a temperature of around 33° C. and the fuel-catalyst was vacuum filtered. For the next step, 22 mL of the resulting fuel was put in contact with 0.6 g of the same adsorbent under the same conditions and process, producing a fuel with 370 ppm of sulfur. Thereafter, a third sulfur adsorption with 6 mL of the product from the previous step and 1.2 g of Cu—Na—Y Zeolite was performed following the same procedure. The resulting stream fuel concentration was 133 ppm.

### Example 4. Multi-Step Desulfurization of Jet Fuel with Copper and Cobalt Ion Exchanged on Na—Y Zeolite Series

In this Example, Applicants demonstrate the desulfurization of jet fuel by utilizing copper and cobalt ion-exchanged Na—Y Zeolites (Cu—Na—Y Zeolite and Co—Na—Y Zeolite, respectively) as adsorbents. The zeolites were wet ion exchanged. The zeolites are also referred to as catalysts.

### Example 4.1. Catalyst Preparation

About 20 g of cobalt nitrate (Co(NO₃)₂.6H₂O) was added to 150 mL of DI water and stirred for 5 minutes until the salt completely dissolved. Next, 12 g of Na—Y zeolite was added to the solution and was left to ion exchange stirring for 24 hours. The solution was washed, vacuum filtered and placed in a Petri dish to dry at 105° C. for 9 hours. The catalyst was calcined at 450° C. for 8 hours. The ion exchanged Co—Na—Y Zeolite was then ready to be used. In addition to the Co—Na—Y Zeolite catalyst, the Cu—Na—Y Zeolite from Example 3 was also used in this Example.

### Example 4.2. Sulfur Adsorption

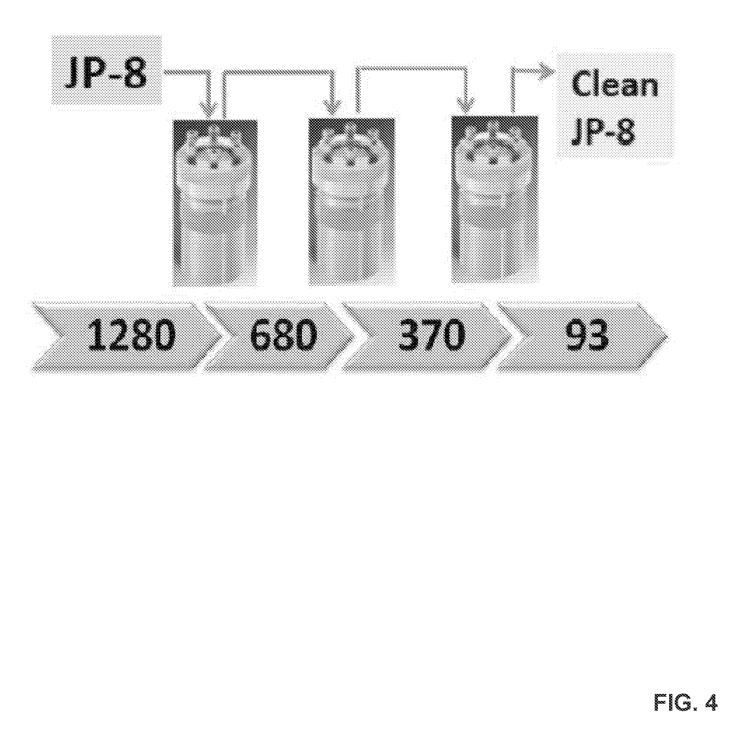

The series of Example 4 consists of a four step desulfurization using Cu—Na—Y Zeolite and Co—Na—Y Zeolite as catalysts (FIG. 4). 27 mL of JP-8 containing 1,280 ppm of sulfur (sulfides, disulfides, thiols, thiophenes, benzothiophenes and dibenzothiophenes) and 0.74 g of Cu—Na—Y Zeolite were put in contact in the reactor vessel. The reactor was properly sealed and placed in the oven (oven temperature=176° C.) for 3 hours. The reactor was cooled to a temperature around 33° C. and the fuel-catalyst mixture was vacuum filtered. The catalyst free fuel was sent to test (680 ppm sulfur) and also stored for a next desulfurization step. For the next step, 22 mL of the resulting fuel was put in contact with 0.6 g of the same adsorbent under the same conditions and process, producing a fuel with 370 ppm of sulfur. Thereafter, a third sulfur adsorption with 6 mL of the product from the previous step and 1.2 g of Co—Na—Y Zeolite was carried following the same procedure, resulting in a 93 ppm product stream (92.7% reduction).

### Example 5. Single-Step Desulfurization of Motor and Aviation Fuel with Cu—Na—Y Zeolite and Ag—Na—Y Zeolite Wet Impregnated Catalysts

This example provides a single-step method for the desulfurization of motor and aviation fuel using Cu—Na—Y Zeolite and wet impregnated Ag—Na—Y Zeolites. The zeolites are also referred to as catalysts.

### Example 5.1. Catalyst Preparation

Ag—Na—Y Zeolites (3% Ag wet impregnated) were prepared as follows. 0.2-0.3 g silver nitrate (AgNO₃) was added to 15-25 mL of DI water and stirred for 5-10 minutes until the salt completely dissolved. 5-6 g of Na—Y Zeolite was added to the solution and was left to stir for an additional 15-25 minutes. The solution was dried in an oven at 90-120° C. for 24 hours. The catalyst was calcined at 430-480° C. for 2-4 hours in helium gas. The formed 3% Ag/Na—Y Zeolite catalyst was then ready to be used.

Wet impregnated Cu—Na—Y Zeolites were prepared by a method similar to Ag—Na—Y Zeolite.

### Example 5.2. Desulfurization Conditions

The sulfur in the motor and aviation fuel was around 1,100-1,300 ppm. The motor and aviation fuel/Catalyst was 30-40 mL/g. The oven temperature was 150-200° C. for 2-4 hours. A batch reactor was used. Table 1 shows the results for the single-step adsorption using different materials.

TABLE 1
Single-step sulfur adsorption on different materials.

| Sample | % Reduction in S |
|---|---|
| 1% Cu—Na—Y Zeolite | 30.3 |
| 3% Cu—Na—Y Zeolite | 35.9 |
| 9% Cu—Na—Y Zeolite | 36.9 |
| 3% Ag—Na—Y Zeolite | 41.3 |

### Example 6. Multi-Step Desulfurization of Motor and Aviation Fuels with Copper and Nickel Ion Exchanged Na—Y Zeolite Series

This example provides a multi-step method for the desulfurization of motor and aviation fuel using ion exchanged Cu—Na—Y Zeolite and ion exchanged Ni—Na—Y Zeolite. The zeolites are also referred to as catalysts.

### Example 6.1. Catalyst Preparation

About 20-30 g of metal nitrate was added to 120-250 mL of DI water and stirred for 5-10 minutes until the salt completely dissolved. About 5-10 g of Na—Y Zeolite was added to the solution that was left to ion exchange stirring for 22-25 hours. The solution was washed, vacuum filtered, and dried in an oven at 90-120° C. for 8-10 hours. The catalyst was calcined at 430-480° C. for 8-10 hours. The ion exchanged Cu—Na—Y Zeolite was then ready to be used.

### Example 6.2. Sulfur Adsorption

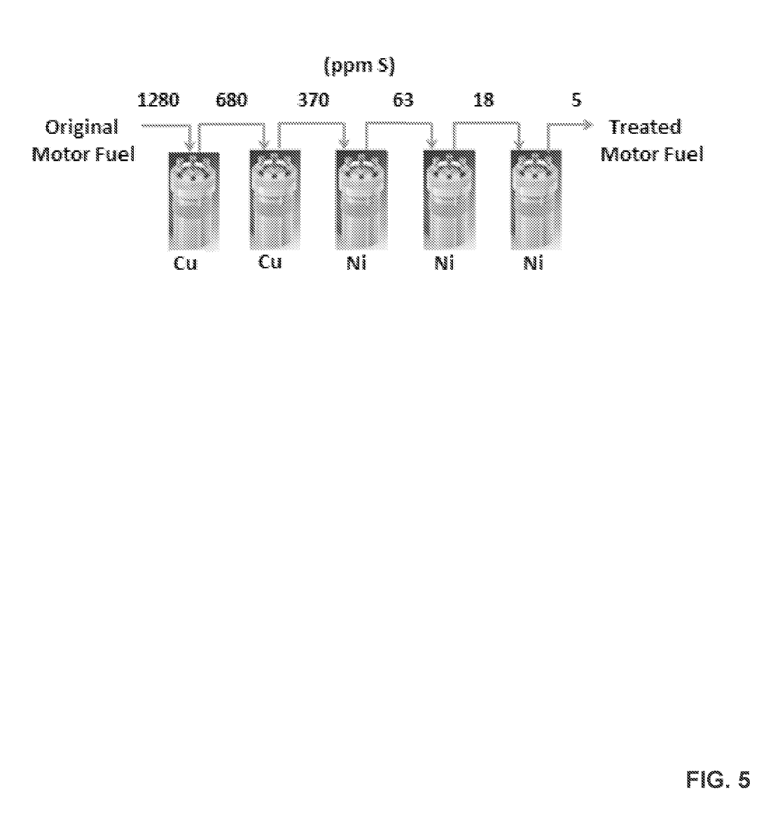

The series in this example consists of four sequential desulfurization steps using Cu—Na—Y Zeolite and Ni—Na—Y Zeolite catalysts, as shown in FIG. 5. 25-30 mL of motor and aviation fuel containing 1,000-1,300 ppm of sulfur (i.e., sulfides, disulfides, thiols, thiophenes, benzothiophenes and dibenzothiophenes) and 0.6-1.0 g of Cu—Na—Y Zeolite were put in contact in the reactor vessel. The reactor was properly sealed and placed in the oven (oven temperature=150-200° C.) for 2-4 hours.

The reactor was cooled to a temperature around 25-35° C. and the fuel-catalyst mixture was vacuum filtered. The treated fuel contained around 680 ppm of sulfur. For the next step, 20-30 mL of the resulting supernatant was put in contact with 0.5-1.0 g of the same adsorbent (fresh) under the same conditions, producing a fuel with around 370 ppm of sulfur. Thereafter, in the third step, sulfur adsorption was carried out with 5-10 mL of the fuel from the previous step and 1-1.5 g of Ni—Na—Y Zeolite, resulting in 63 ppm of sulfur. The same approach was used for the fourth and fifth steps. The final sulfur concentration in treated motor and aviation fuel was 5 ppm. After five treatments with Cu—Cu—Ni—Ni—Ni catalysts, the total S content was reduced by 99.6%.

### Example 7. Multi-Step Desulfurization of Motor and Aviation Fuel Using Ion Exchanged Cu—Na—Y Zeolite and Wet Impregnated 4.5% Ag-4.5%-Cu—Na—Y Zeolites

This example provides a multi-step method for the desulfurization of motor and aviation fuel using ion exchanged Cu—Na—Y Zeolite and wet impregnated 4.5% Ag-4.5% Cu—Na—Y Zeolite (Ag—Cu—Na—Y Zeolite). The zeolites are also referred to as catalysts.

### Example 7.1. Catalyst Preparation

The ion exchanged Cu—Na—Y Zeolites were prepared by the method outlined in Example 6. The wet impregnated Ag—Cu—Na—Y Zeolites were prepared by the method outlined in Example 5.

### Example 7.2. Desulfurization Conditions (3 Steps)

The Sulfur in the motor and aviation fuel was around 1,100-1,300 ppm. The motor and aviation fuel/Catalyst value was 30-40 mL/g for the first two steps, and 3-7 mL/g for the third step. The oven temperature was 150-200° C. for 2-4 hours. A batch reactor was utilized.

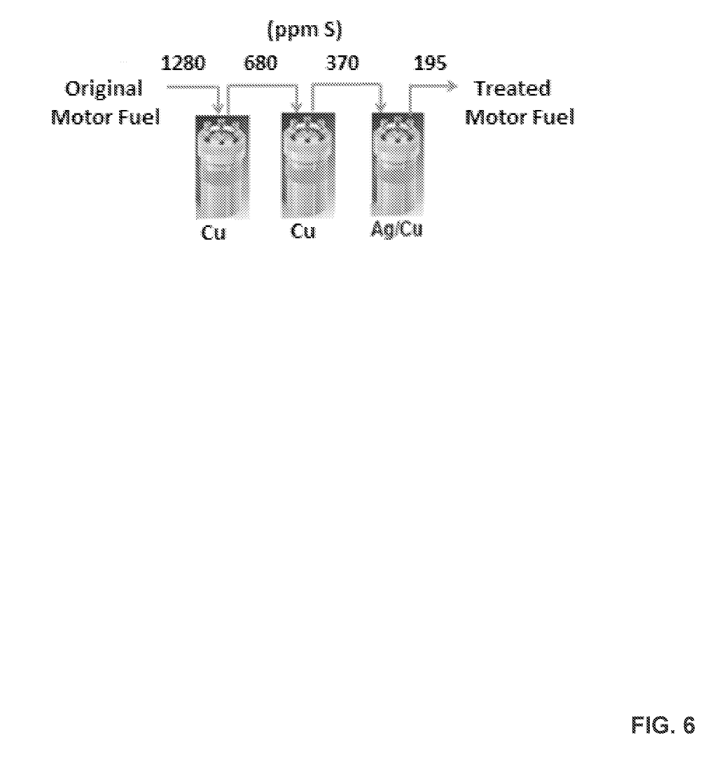

The total sulfur adsorption resulted in an overall 84.8% sulfur reduction. The sulfur content after each step can be seen in FIG. 6.

### Example 8. Desulfurization of Motor and Aviation Fuel Using γ-Al₂O₃Nanowires as Adsorbent Support

This example provides a single-step method for the desulfurization of motor and aviation fuel using γ-Al₂O₃ nanowires as the adsorbent support. The adsorbent support is also referred to as the catalyst support.

### Example 8.1. Catalyst Preparation

The catalysts prepared are listed in Table 2. The same methods outlined in Examples 5-6 were utilized to prepare these catalysts.

TABLE 2
Single-step sulfur adsorption on γ-Al₂O₃ NW based-materials.

| Sample | % Reduction in sulfur |
|---|---|
| Calcined γ-Al₂O₃ NW | 8.6 |
| 3% Cu/γ-Al₂O₃ NW | 19.5 |
| 9% Cu/γ-Al₂O₃ NW | 8.6 |

### Example 8.2. Desulfurization Conditions

The sulfur in the motor and aviation fuel was around 1,100-1,300 ppm. The motor and aviation fuel/Catalyst was 30-40 mL/g for the 2 first steps, and 3-7 mL/g for the third step. The oven temperature was 150-200° C. for 2-4 hours. A batch reactor was used. Table 2 shows the results for the single-step adsorption on γ-Al₂O₃ NW based-materials.

### Example 9. Surface Areas of Selected Adsorbents

This example summarizes the surface areas of various adsorbents. The surface areas are summarized in Table 3.

TABLE 3
The BET surface areas of various adsorbents.

| | Surface area |
|---|---|
| Material | (m²/g) |
| 3% Cu—Y Zeolite | 433 |
| 9% Cu—Y Zeolite | 188 |
| 3% Ag—Y Zeolite | 623 |
| Cu—Y Zeolite | 623 |
| Ni—Y Zeolite | 596 |

### Example 10. Desulfurization of Motor and Aviation Fuel at Room Temperature

The desulfurization can also be held at room temperature, as it is shown in the embodiments herein.

### Example 10.1. Desulfurization Conditions

The motor and aviation fuel/adsorbent ratio was about 30-40 mL/g. The sulfur in the motor and aviation fuel was around 1,100-1,300 ppm. Desulfurization was conducted in a batch reactor while stirring for 12-15 hours. The results are summarized in Table 3.

TABLE 4
Results for the desulfurization of motor and aviation fuel.

| Sample | % Reduction in S |
|---|---|
| Na—Y Zeolite | 2.3 |
| 3% Ag—Y Zeolite | 15.3 |
| (wet impregnated) | |
| 9% Ag/γ-Al₂O₃ | 9.4 |
| NW | |

### Example 11. Dodecane and Toluene Desulfurization by Ion Exchanged Cu—Na—Y Zeolites

This example summarizes the desulfurization of dodecane and toluene from motor and aviation fuels by ion exchanged Cu—Na—Y Zeolites that were described in Example 6.

### Example 11.1. Desulfurization Conditions (Single-Step)

The model fuels prepared for the following desulfurization experiments contained a sulfur concentration of about 400 ppmw each. The fuel/adsorbent ratio was 15 mL/g. The oven temperature was from 80-120° C. A batch reactor was utilized for about 2-4 hours. Table 5 shows the results for the single-step adsorption for fine chemicals.

TABLE 5
Single step sulfur adsorption for fine chemicals.

| Hydrocarbon | % Reduction in sulfur |
|---|---|
| Toluene | 51.6 |
| n-Dodecane | 98.8 |
| 5 wt % Toluene + | 96.3 |
| 95 wt % n-Dodecane | |

### Example 12. One-Step Desulfurization of Motor and Aviation Fuel Using Ion Exchanged Cu—, Ni—, and Co—Na—Y Zeolites

This example illustrates a one-step desulfurization of motor and aviation fuels by using the following ion exchanged zeolites that were described in Example 6: Cu—Na—Y Zeolites, Ni—Na—Y Zeolites, and Co—Na—Y Zeolites.

### Example 12.1. Desulfurization Conditions

The sulfur in the motor and aviation fuel was around 1,100-1,300 ppm. The motor and aviation fuel/adsorbent content was 30-40 mL/g. The oven temperature was from about 150-200° C. A batch reactor was utilized at that temperature for 2-4 hours. Table 6 summarizes the results of the one-step desulfurization with the above mentioned materials.

TABLE 6
One-step desulfurization with Cu—, Ni, and Co ion
exchanged materials, including Cu—Na—Y Zeolites
(Cu IE), Ni—Na—Y Zeolites (Ni IE), and Co—Na—Y
Zeolites (Co IE).

| Catalyst | % Reduction in sulfur |
|---|---|
| Cu IE | 46.8 |
| Ni IE | 18.2 |
| Co IE | 8.7 |

Ni and Co-ion exchanged materials show better desulfurization when placed after Cu ion exchanged materials in a series, but they show lower sulfur removal if used in the first step (Table 6). The above mentioned examples demonstrate that a single material may not be efficient for all steps. Rather, the most efficient series includes a combination of different materials in a specific sequence for selective adsorption of the sulfur compounds.

### Example 13. Adsorption of Nitrogen Compounds with Cu—Na—Y Zeolites

This example illustrates adsorption of nitrogen compounds from motor and aviation fuels by ion exchanged Cu—Na—Y Zeolites that were described in Example 6.

### Example 13.1. Denitrogenation Conditions

The motor and aviation fuel/adsorbent content was 30-40 mL/g. The oven temperature was from about 150-200° C. A batch reactor was utilized at that temperature for 2-4 hours. The total nitrogen content in motor and aviation fuel before adsorption was 10 ppm. After treatment with Cu—Na—Y Z, it was reduced to 0.9 ppm, a 91% nitrogen reduction in one step.

### REFERENCES

1. EPA. http://epa.gov/airtrends/sixpoll.html. 2015.
2. Sydbom, A., Blomberg, A., Parnia, S., Stenfors, N., Sandstrom, T., and Dahlen, S. E., Health effects of diesel exhaust emissions. European Respiratory Journal, 2001. 17(4): p. 733-746.
3. Agency, E. P., http://www2.epa.gov/laws-regulations. 2015.
4. Corro, G., Sulfur impact on diesel emission control—A review. Reaction Kinetics and Catalysis Letters, 2002. 75(1): p. 89-106.
5. Yang, W. M., Liu, L. K., Zhou, Z. P., Qiu, C. X., Ma, P. F., Liu, H., and Xu, W. Z., Rational design and preparation for novel denitrogenation adsorbents by computational simulation and improved atom transfer radical polymerization. New Journal of Chemistry, 2013. 37(9): p. 2758-2767.
6. Ahmed, I. and Jhung, S. H., Adsorptive denitrogenation of model fuel with CuCl-loaded metal-organic frameworks (MOFs). Chemical Engineering Journal, 2014. 251: p. 35-42.
7. Rashidi, S., Nikou, M. R. K., and Anvaripour, B., Adsorptive desulfurization and denitrogenation of model fuel using HPW and NiO-HPW modified aluminosilicate mesostructures. Microporous and Mesoporous Materials, 2015. 211: p. 134-141.
8. Zheng, Y. and Chou, L., Adsorbents for denitrogenation desulfurization of hydrocarbon oils and methods of producing. 2011, U.S. Pat. No. 7,935,248.
9. K. Liu, C.S.S.a.S.V., Hydrogen and Syngas Production and Purification Technologies. 2009, Hoboken, N.J.: John Wiley & Sons, Inc.
10. Ma, X. L., Sakanishi, K. Y., and Mochida, I., HYDRODESULFURIZATION REACTIVITIES OF VARIOUS SULFUR-COMPOUNDS IN DIESEL FUEL. Industrial & Engineering Chemistry Research, 1994. 33(2): p. 218-222.
11. Barrai, F. and Castaldi, M. J., Experimental Investigation of a JP8 Fuel Processor: Autothermal Reformer and CO-Cleanup Train. Industrial & Engineering Chemistry Research, 2010. 49(4): p. 1577-1587.
12. Velu, S., Ma, X. L., and Song, C. S., Selective adsorption for removing sulfur from jet fuel over zeolite-based adsorbents. Industrial & Engineering Chemistry Research, 2003. 42(21): p. 5293-5304.
13. Srivastava, V. C., An evaluation of desulfurization technologies for sulfur removal from liquid fuels. Rsc Advances, 2012. 2(3): p. 759-783.
14. Takahashi, A., Yang, F. H., and Yang, R. T., New sorbents for desulfurization by pi-complexation: Thiophene/benzene adsorption. Industrial & Engineering Chemistry Research, 2002. 41(10): p. 2487-2496.
15. Hernandez-Maldonado, A. J. and Yang, R. T., Desulfurization of liquid fuels by adsorption via pi complexation with Cu(I)-Y and Ag-Y zeolites. Industrial & Engineering Chemistry Research, 2003. 42(1): p. 123-129.
16. Hernandez-Maldonado, A. J., Yang, R. T., and Cannella, W., Desulfurization of commercial jet fuels by adsorption via pi-complexation with vapor phase ion exchanged Cu(I)-Y zeolites. Industrial & Engineering Chemistry Research, 2004. 43(19): p. 6142-6149.
17. Xu, X. H., Zhang, S. Y., Li, P. W., and Shen, Y. S., Adsorptive desulfurization of liquid Jet-A fuel at ambient conditions with an improved adsorbent for on-board fuel treatment for SOFC applications. Fuel Processing Technology, 2014. 124: p. 140-146.
18. Bailey, G. W. and Swan, G. A., Nickel adsorbent for sulfur removal from hydrocarbon feeds. 1987, U.S. Pat. No. 4,634,515.
19. Morris, M., Robinson, S. C. F., and Lander, D. F., Adsorbents for separating nitrogen from a feed gas. 1999, U.S. Pat. No. 5,993,516.
20. Bartek, R., Removal of sulfur from a hydrocarbon stream by low severity adsorption. 1999, U.S. Pat. No. 5,919,354.
21. Song, C., Ma, X., Sprague, M. J., and Subramani, V., Deep desulfurization of hydrocarbon fuels. 2003, WO 2003/068892.
22. Cutler, W., He, L., Owens, L., and Sorensen, C., Structured adsorbents for desulfurizing fuels. 2004, U.S. App. No. 2004/0118747.
23. J, H. M. A. and T, Y. R., Denitrogenation of liquid fuels. 2005, WO 2005/075608.
24. Takatsu, K. and Takegoshi, G., Adsorbent for removing sulfur compound, process for producing hydrogen and fuel cell system. 2005, European Patent No. EP 1 550 505.
25. Antonio, J. A., Jacome, M. C., Tones, G., Chavez, C., Ortiz, L., de Lourdes Mosqueira Mondragon, M., Salinas, E., Aguilar, J., Vallejo, R. M., and Ramirez, F., Selective adsorbent material and its use. 2005, U.S. App. No. 2005/0263441.
26. Yang, R. T., Hernandez-Maldonado, A. J., Yang, F. H., and Takahashi, A., Selective sorbents for purification of hydrocarbons. 2006, U.S. Pat. No. 7,094,333.
27. Zulauf, G. B. and Rohrbach, R. P., Portable fuel desulfurization unit. 2010, U.S. Pat. No. 7,704,383.
28. Toida, Y., Method of desulfurizing hydrocarbon oil. 2011, U.S. Pat. No. 8,021,540.
29. Richter, I., Kiener, C., Malkowsky, I. M., Achmann, S., Hagen, G., Haemmerle, M., and Moos, R., Method for removing compounds containing sulfur from fuels. 2011, U.S. Pat. App. No. 2011/0138781.
30. Wei, X., Husson, S. M., Mello, M. V. D., Chinn, D., and He, Z., Adsorption of dibenzothiophenes from hydrocarbon and model diesel feeds. 2012, U.S. Pat. No. 8,142,647.
31. Ratnasamy, C. C., Wagner, J. P., Spivey, R. S., and Anfang, H. G., Desulfurization system and method for desulfurizing a fuel stream. 2012, U.S. Pat. No. 8,323,603.
32. Novak, W. J. and GATT, J. E., Low temperature adsorbent for removing sulfur from fuel. 2013, WO 2013/043629.
33. Velu, S., Song, C. S., Engelhard, M. H., and Chin, Y. H., Adsorptive removal of organic sulfur compounds from jet fuel over K-exchanged NiY zeolites prepared by impregnation and ion exchange. Industrial & Engineering Chemistry Research, 2005. 44(15): p. 5740-5749.
34. Hernandez-Maldonado, A. J., Yang, F. H., Qi, G., and Yang, R. T., Desulfurization of transportation fuels by pi-complexation sorbents: Cu(I)-, Ni(II)-, and Zn(II)-zeolites. Applied Catalysis B-Environmental, 2005. 56(1-2): p. 111-126.
35. Ma, X. L., Velu, S., Kim, J. H., and Song, C. S., Deep desulfurization of gasoline by selective adsorption over solid adsorbents and impact of analytical methods on ppm-level sulfur quantification for fuel cell applications. Applied Catalysis B-Environmental, 2005. 56(1-2): p. 137-147.
36. Velu, S., Ma, X. L., Song, C. S., Namazian, M., Sethuraman, S., and Venkataraman, G., Desulfurization of JP-8 jet fuel by selective adsorption over a Ni-based adsorbent for micro solid oxide fuel cells. Energy & Fuels, 2005. 19(3): p. 1116-1125.
37. Xu, X., Zhang, S., Li, P., and Shen, Y., Equilibrium and kinetics of Jet-A fuel desulfurization by selective adsorption at room temperatures. Fuel, 2013. 111: p. 172-179.
38. Wang, Y., Latz, J., Dahl, R., Pasel, J., and Peters, R., Liquid phase desulfurization of jet fuel by a combined pervaporation and adsorption process. Fuel Processing Technology, 2009. 90(3): p. 458-464.
39. Wang, Y., Geder, J., Schubert, J. M., Dahl, R., Pasel, J., and Peters, R., Optimization of adsorptive desulfurization process of jet fuels for application in fuel cell systems. Fuel Processing Technology, 2012. 95: p. 144-153.
40. Nejad, N. F., Shams, E., Amini, M. K., and Bennett, J. C., Synthesis of magnetic mesoporous carbon and its application for adsorption of dibenzothiophene. Fuel Processing Technology, 2013. 106: p. 376-384.
41. Hernandez-Maldonado, A. J. and Yang, R. T., Desulfurization of transportation fuels by adsorption. Catalysis Reviews-Science and Engineering, 2004. 46(2): p. 111-150.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of removing heteroatoms from a fluid, wherein the method comprises:
associating the fluid with one or more adsorbents at a desired temperature or temperature range,
wherein the associating results in the removal of the heteroatoms from the fluid at the desired temperature or temperature range.

2-3. (canceled)

4. The method of claim 1, wherein the associating occurs in a single step or in multiple steps by contacting the fluid with one or more adsorbents.

5. (canceled)

6. The method of claim 1, wherein the associating occurs by associating the fluid with a plurality of adsorbents in a sequential manner,
wherein the sequential association is arranged to maximize heteroatom removal,
wherein the plurality of adsorbents are sequenced in a specific order to selectively remove competing heteroatoms from fluid components, and
wherein heteroatom removal efficacy is maximized by requiring less adsorbents, requiring less processing time, enhancing heteroatom removal efficiency, removing more heteroatoms, or combinations thereof.

7-9. (canceled)

10. The method of claim 1, wherein the heteroatoms comprise heteroatom-containing compounds selected from the group consisting of sulfur-containing compounds, nitrogen-containing compounds, oxygen-containing compounds, sulfides, disulfides, thiols, mercaptans, hydrogen sulfides, thiophenes, benzothiophenes, dibenzothiophenes, anilines, pyrroles, indoles, carbazoles, phenols, alcohols, acids, and combinations thereof.

11-14. (canceled)

15. The method of claim 1, wherein the fluid is selected from the group consisting of fuels, hydrocarbons, alcohols, water, organic solvents, gaseous state fluids, liquid state fluids, hydrocarbon fuel, liquid state hydrocarbon fuel, gaseous state hydrocarbon fuel, jet fuel, diesel fuel, kerosene, gasoline, natural gas, hydrocarbon fine chemical, and combinations thereof.

16-18. (canceled)

19. The method of claim 1, wherein the fluid is a gaseous state hydrocarbon fuel.

20-21. (canceled)

22. The method of claim 1, wherein the fluid has a total heteroatom content that ranges from about 1 ppmw to about 5000 ppmw, a total sulfur content of 3000 ppmw or greater, and a total nitrogen content of 500 ppmw or greater, and
wherein the removing results in a reduction of heteroatoms in the fluid by more than about 50%, or by more than about 99%.

23-24. (canceled)

25. The method of claim 1, wherein the one or more adsorbents are the same.

26. The method of claim 1, wherein the one or more adsorbents are different.

27. The method of claim 1, wherein the one or more adsorbents are selected from the group consisting of activated carbon, zeolites, ion exchanged zeolites, ion impregnated zeolites, alumina, alumina nanowires, carbon-based supports, and combinations thereof.

28. The method of claim 1, wherein the one or more adsorbents comprise additional components, wherein the additional components are selected from the group consisting of active metals, transition metals, Co, Cu, Ce, Ni, Fe, Mn, Pd, Ag, W, Zn, Pt, Au, Cr, V, Ti, Mo, oxides thereof, sulfides thereof, and combinations thereof.

29. (canceled)

30. The method of claim 1, wherein the one or more adsorbents comprise H or metals selected from the group consisting of K, Na and combinations thereof.

31. The method of claim 1, wherein the one or more adsorbents comprises a single transition metal or a plurality of transition metals.

32-33. (canceled)

34. The method of claim 1, wherein one or more adsorbent components are affixed to a solid support, wherein the solid support is selected from the group consisting of alumina, alumina nanowires, activated carbon, zeolites, and combinations thereof.

35. (canceled)

36. The method of claim 1,
wherein the one or more adsorbents are selected from the group consisting of ion exchanged zeolites, ion impregnated zeolites, wet impregnated adsorbents, and combinations thereof; and
wherein the one or more adsorbents have a surface area of at least 50 m²/g to about 1,000 m²/g.

37-41. (canceled)

42. The method of claim 1, wherein the desired temperature or temperature range comprises temperatures from about 10° C. to about 500° C., from about 10° C. to about 25° C., above 100° C., from about 100° C. to about 250° C., or from about 150° C. to about 500° C.

43-46. (canceled)

47. The method of claim 1, wherein the removing occurs without the addition or utilization of any non-oxygen gases, reactive gases, or H₂.

48-49. (canceled)

50. The method of claim 1, wherein the removing reduces the heteroatom content of the fluid to below 30 ppmw or to below 10 ppmw.

51-56. (canceled)

57. The method of claim 1, wherein the heteroatoms are removed from chemical components contained in the fluid, and wherein different heteroatoms are removed simultaneously.

58-59. (canceled)

60. The method of claim 1, wherein heteroatom removal occurs selectively.
